(19) United States
(12) Patent Application Publication
(10) Pub. No.: US 2002/0055637 A1
(43) Pub. Date: May 9, 2002

(54) Methods for synthesis of amino-tetrahydroisoquinoline-carboxylic acids

(76) Inventors: Song Liu, San Diego, CA (US); William Martin Rennells, Schenectady, NY (US)

Correspondence Address: DINSMORE & SHOHL, LLP, 1900 CHEMED CENTER, 255 EAST FIFTH STREET, CINCINNATI, OH, 45202, US

(21) Appl. No.: 10/036,671

(22) Filed: Dec. 21, 2001

(51) Int. Cl. C07D 215/00

(52) U.S. Cl. 546/152

(57) ABSTRACT

Methods of preparing an amino-substituted-tetrahydroisoquinoline-carboxylic acid include the steps of providing a support-bound amino-substituted-tetrahydroisoquinoline-carboxylate wherein the ring nitrogen has a protecting group; attaching a first moiety to the amino-substituted group of the support-bound amino-substituted-tetrahydroisoquinoline-carboxylate; de-protecting the ring nitrogen; attaching a second moiety to the ring nitrogen to form a support-bound intermediate; and cleaving the support-bound intermediate from the solid support to form the carboxylic acid.

### FIELD OF INVENTION

The present invention relates to methods of preparing amino-tetrahydroisoquinoline-carboxylic acids. More particularly, the invention relates to the solid phase synthesis of amino-substituted-tetrahydroisoquinoline-carboxylates using a solid support. The invention also relates to methods of preparing combinatorial libraries of amino-substituted-tetrahydroisoquinoline-carboxylic acids.

### BACKGROUND ART

The amino-substituted-tetrahydroisoquinoline-carboxylic acids (amino-substituted-TIQ-carboxylate acids) are useful in numerous pharmaceutical applications. Such compounds have been useful in treatment of degenerative joint disorders, disorders of the connective tissue, ulcerations, arteriosclerosis, stenosis, inflammation, carcinomatosis, anorexia, and septic shock. Thus, it is desirable to generate amino-substituted-TIQ-carboxylic acids for testing as potential drug candidates. The acceleration of drug discovery has generated growing demands for efficient synthetic methods to produce therapeutic candidates. Preferably the methods are suitable for use in generating combinatorial libraries.

Schudok, U.S. Pat. No. 5,962,471, teach substituted 6- and 7-amino-tetrahydroisoquinoline-carboxylic acids suitable for therapy of disorders involving increased activity of matrix-degrading metalloproteinases. Schudok teaches that methods of synthesizing such compounds include the nitration of tetrahydroisoquinoline; acylation with carbonyl or sulfonyl chloride, carboxylic or sulfonic imidazolides, chloroformic acid esters, active esters or anhydrides; treatment with an amino acid, carboxylic acid, aldehyde or substituted guanidine; or alkylation. Grunewald et al., J Med. Chem., 42:1982-1990 (1999), teach that the enzyme phenylethanolamine N-methyltransferase may be inhibited by 3,7-disubstituted-1,2,3,4-tetrahydroisoquinolines. Grunewald et al. teach synthetic methods for tetrahydroisoquinolines which include steps such as the treatment of phenylamine with formaldehyde and hydrochloric acid, and nitration of tetrahydroisoquinoline-carboxylic acid by treatment with nitronium tetrafluoroborate and acetonitrile. Grunewald et al. further teach that electrophillic substitution reactions such as nitration, chlorosulfonation and Friedel-Crafts acylation have been successful in introducing substituents on the 7- position of tetrahydroisoquinoline. Nouvet et al., Tetrahedron, 55:4685-4698 (1999), teach the synthesis of perhydrodiazepinones as peptidomimetics. Nouvet et al. teach that t-butyl esters may be cleaved by treatment of HBr and the mixture of acetic acid/trifluoroacetic acid as a solvent.

There is a need for facile and efficient methods for the synthesis of amino-substituted-TIQ-carboxylic acids. It is desirable that the methods conveniently produce combinatorial libraries of compounds.

### SUMMARY OF THE INVENTION

It is therefore an object of the invention to provide novel methods of preparing amino-tetrahydroisoquinoline-carboxylic acids, particularly amino-substituted-tetrahydroisoquinoline-carboxylic acids. It is also an object of the invention to provide novel methods of preparing combinatorial libraries of amino-substituted-tetrahydroisoquinoline-carboxylic acids.

In accordance with one aspect of the invention, there are provided methods of preparing an amino-substituted-tetrahydroisoquinoline-carboxylic acid having the structure:

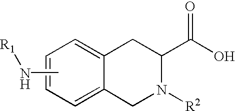

wherein R¹ is alkyl, aryl, heterocyclic moiety, amide, sulfonamide, urea, thiourea, or alcohol and R² is alkyl, aryl, heterocyclic moiety, amide, sulfonamide, urea, thiourea, hydrogen, or alcohol. The methods comprise the steps of providing an orthogonally protected amino-substituted-tetrahydroisoquinoline-carboxylic acid wherein the orthogonally protected amino-substituted-tetrahydroisoquinoline-carboxylic acid is attached to a solid support and therefore is in the form of a carboxylate; attaching the R¹ group to the orthogonally protected amino-substituted-tetrahydroisoquinoline-carboxylate; de-protecting the orthogonally protected amino-substituted-tetrahydroisoquinoline-carboxylate to form a deprotected, R¹-substituted-amino-substituted-tetrahydroisoquinoline-carboxylate; attaching the R² group to the ring nitrogen of the R¹-substituted-amino-substituted-tetrahydroisoquinoline-carboxylate to form an intermediate; and cleaving the intermediate from the solid support to form an amino-substituted-tetrahydroisoquinoline-carboxylic acid.

In accordance with another aspect of the invention, there are provided methods of preparing a combinatorial library of amino-substituted-tetrahydroisoquinoline-carboxylic acids having the structure:

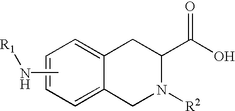

wherein R¹ is alkyl, aryl, heterocyclic moiety, amide, sulfonamide, urea, thiourea, or alcohol and R² is alkyl, aryl, heterocyclic moiety, amide, sulfonamide, urea, thiourea, alcohol, or hydrogen. The methods comprise the steps of: (a) providing an orthogonally protected amino-substituted-tetrahydroisoquinoline-carboxylic acid wherein the orthogonally protected amino-substituted-tetrahydroisoquinoline-carboxylic acid is attached to a solid support and therefore in the form of a carboxylate; (b) attaching a first R¹ group to a first portion of orthogonally protected amino-substituted-tetrahydroisoquinoline-carboxylate and attaching a second R¹ group to a second portion of orthogonally protected amino-substituted-tetrahydroisoquinoline-carboxylate; (c) de-protecting the first and second portions of orthogonally protected amino-substituted-tetrahydroisoquinoline-carboxylate to form first and second deprotected amino-substituted-tetrahydroisoquinoline-carboxylates; (d) attaching a first R₂ group to the ring nitrogen of the first deprotected amino-substituted-tetrahydroisoquinoline-carboxylate to form a first intermediate and attaching a second R² group to the ring nitrogen of the second deprotected amino-substituted-tetrahydroisoquinoline-carboxylate to form a second intermediate; and (e) cleaving the first and second intermediates from the solid support to form first and second substituted amino-substituted-tetrahydroisoquinoline-carboxylic acids. At least one of the first R¹ group and the first R² group is different from the second R¹ group and the second R² group, respectively.

In accordance with another aspect of the invention, there are provided methods of preparing an amino-substituted-tetrahydroisoquinoline-carboxylic acid comprising the steps of providing a support-bound amino-substituted-tetrahydroisoquinoline compound having the structure:

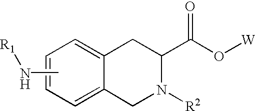

wherein R¹ is alkyl, aryl, heterocyclic moiety, amide, sulfonamide, urea, thiourea, or alcohol and R² is alkyl, aryl, heterocyclic moiety, amide, sulfonamide, urea, thiourea, alcohol, or hydrogen, and W represents the support, and cleaving the support-bound amino-substituted-tetrahydroisoquinoline compound to form a amino-substituted-tetrahydroisoquinoline-carboxylate.

In accordance with a further aspect of the invention, there are provided methods of preparing an amino-substituted-tetrahydroisoquinoline-carboxylic acid comprising the steps of providing a support-bound amino-substituted-tetrahydroisoquinoline-carboxylate wherein the ring nitrogen of the support-bound amino-substituted-tetrahydroisoquinoline-carboxylic acid has a protecting group; attaching a first moiety to the amino-substituted-tetrahydroisoquinoline-carboxylate group of the support-bound amino-substituted-tetrahydroisoquinoline-carboxylate; deprotecting the ring nitrogen; attaching a second moiety to the ring nitrogen to form a support-bound intermediate; and cleaving the support-bound intermediate from the solid support.

In accordance with yet another aspect of the invention, there are provided methods of preparing amino-substituted-tetrahydroisoquinoline-carboxylic acids comprising the steps of providing a support-bound amino-substituted-tetrahydroisoquinoline-carboxylate; attaching a first moiety to a non-ring amino group of the support-bound amino-substituted-tetrahydroisoquinoline-carboxylate; attaching a second moiety to a ring nitrogen of the support-bound amino-substituted-tetrahydroisoquinoline-carboxylate to form a support-bound intermediate; and cleaving the support-bound intermediate from the solid support to form an amino-substituted-tetrahydroisoquinoline-carboxylic acid.

The present invention provides convenient means for producing amino-substituted-tetrahydroisoquinoline-carboxylic acids, and preparing combinatorial libraries thereof. The present invention also provides convenient means for producing 7-amino-tetrahydroisoquinoline-carboxylic acids, and preparing combinatorial libraries thereof. These and additional objects and advantages will be more fully apparent in view of the following description.

### DETAILED DESCRIPTION

As used herein unless specified otherwise, “alkyl” means a hydrocarbon chain which is branched, linear or cyclic, saturated or unsaturated (but not aromatic), substituted or unsubstituted. The term “alkyl” may be used alone or as part of another word where it may be shortened to “alk” (e.g., in alkoxy, alkacyl). Preferred linear alkyls have from one to about twenty carbon atoms, more preferably from one to about ten carbon atoms, more preferably still from one to about six carbon atoms, still more preferably from one to about four carbon atoms; most preferred are methyl or ethyl. Preferred cyclic and branched alkyls have from three to about twenty carbon atoms, more preferably from three to about ten carbon atoms, more preferably still from three to about seven carbon atoms, still more preferably from three to about five carbon atoms. Preferred cyclic alkyls have one hydrocarbon ring, but may have two, three, or more, fused or spirocycle hydrocarbon rings. Preferred alkyls include unsaturated alkyls with from one to about three double or triple bonds, preferably double bonds; more preferably they are mono-unsaturated with one double bond. Also, preferred alkyls include saturated alkyls. Saturated alkyl are referred to herein as “alkanyl”. Alkyls unsaturated with one or more double bonds (no triple bonds) are referred to herein as “alkenyl”. Alkyls unsaturated with one or more triple bonds are referred to herein as “alkynyl”. Preferred substituents of alkyls include halo, alkyl, aryl, heterocycle, hydroxy, alkoxy, aryloxy, thio, alkylthio, arylthio, amino, alkylamino, arylamino, amide, alkylamide, arylamide, formyl, alkacyl, arylacyl, carboxy and its alkyl and aryl esters and amides, sulfo, alkylsulfo, arylsulfo, sulfino, alkylsulfino, arylsulfino, phospho, alkylphospho, arylphospho, phosphino, alkylphosphino, arylphosphino, nitro, and cyano. Substituents of cycloalkyl also include cycloalkyl, aryl and heterocyclic rings which are fused or spirocycle with the initial cycloalkyl. Unsubstituted alkyls are preferred. An alkyl is bonded to another moiety at the “attaching carbon” of the alkyl. As used herein, “primary alkyl” means that the attaching carbon of the alkyl has two or three hydrogens bonded to it; “secondary alkyl” means that the attaching carbon has one hydrogen bonded to it; and “tertiary alkyl means that the attaching carbon has no hydrogens bonded to it.

As used herein, “heteroatom” means an atom other than carbon, preferably a nitrogen, oxygen, or sulfur atom.

As used herein, “alkylene” means an alkyl which connects two other moieties, “heteroalkylene” means an alkylene having one or more heteroatoms in the connecting chain.

As used herein unless specified otherwise, “aryl” means an aromatic hydrocarbon ring (or fused rings) which is substituted or unsubstituted. The term “aryl” may be used alone or as part of another word (e.g., in aryloxy, arylacyl). Preferred aryls have from six to about fourteen, preferably to about ten, carbon atoms in the aromatic ring(s), and a total of from about six to about twenty, preferably to about twelve, carbon atoms. Preferred aryls are phenyl or naphthyl; most preferred is phenyl (Ph). Preferred substituents of aryl include halo, alkyl, aryl, heterocycle, hydroxy, alkoxy, aryloxy, thio, alkylthio, arylthio, amino, alkylamino, arylamino, amide, alkylamide, arylamide, formyl, alkacyl, arylacyl, carboxy and its alkyl and aryl esters and amides, sulfo, alkylsulfo, arylsulfo, sulfino, alkylsulfino, arylsulfino, phospho, alkylphospho, arylphospho, phosphino, alkylphosphino, arylphosphino, nitro, and cyano. Substituents of aryl also include cycloalkyl and heterocyclic rings which are fused with the aryl ring or rings. Also, unsubstituted aryl are preferred.

As used herein unless specified otherwise, “heterocycle” or “heterocyclic” means a saturated, unsaturated or aromatic cyclic hydrocarbon ring (or fused rings) with one or more heteroatoms in the hydrocarbon ring(s). Preferred heterocycles have from one to about six heteroatoms in the ring(s), more preferably one or two or three heteroatoms in the ring(s). Preferred heterocycles have from three to about fourteen, preferably to about ten, carbon plus heteroatoms in the ring(s), more preferably from three to about seven, more preferably still five or six, carbon plus heteroatoms in the rings(s); and a total of from three to about twenty carbon plus heteroatoms, more preferably from three to about ten, more preferably still five or six, carbon plus heteroatoms. Preferred heterocycles have one ring, but may have two, three, or more, fused rings. More preferred heterocyclic rings include those which are one ring with 5 or 6 carbon plus heteroatoms in the ring with no more than three ring heteroatoms, no more than two of which are O and S. Still more preferred are such 5- or 6-ring atom heterocycles with one or two ring atoms being O or S and the others being C; or with one, two or three ring atoms being N and the others being C. Such preferred 5- or 6-ring atom heterocycles are preferably saturated, unsaturated with one or two double bonds, or aromatic. Such preferred 5- or 6-ring atom heterocycles are preferably a single ring; or fused with a 3- to 6-ring atom hydrocarbon ring which is saturated, unsaturated with one double bond, or aromatic (phenyl); or fused with another such 5- or 6-ring atom heterocyclic ring. Heterocycles are unsubstituted or substituted. Preferred heterocycle substituents are the same as for alkyl.

As used herein, “strong base” means an inorganic hydroxide base, alkyl-alkali metal (e.g., n-butyllithium), alkali metal hydride (e.g., sodium hydride), alkoxide salt (e.g., sodium methoxide), alkali metal amide (e.g., lithium diisopropyl amide), and the like. As used herein, “substantial amount” means a sufficient amount of a specified material such that it effects a subject invention process in a measurable way. As used herein, “substantially free” means a product or other material has less than about 10%, preferably less than about 5%, more preferably less than about 2%, more preferably still less than about 1% of the indicated compound.

As used herein, “non-protic and non-oxidizing solvent” means a solvent that does not dissociate to provide a substantial and measurable proton concentration, and does not have substantial oxidizing potential. Protic solvents include, for example, water, methanol, ethanol, dimethylformamide and the like. Oxidizing solvents include, for example, dimethylsulfoxide, and the like.

As used herein “combinatorial library” of compounds means a mixture of related compounds or a group of individual compounds, made substantially simultaneously by substantially the same process using a mixture of or individual related reactants to obtain related compounds. The combinatorial library may be formed by separating a TIQ-carboxylic acid reactant into at least first and second portions and reacting the respective portions with different R¹- and/or R²-containing reactants. Alternatively, the combinatorial library may be formed by reacting a TIQ-carboxylic acid reactant with a mixture of R¹-containing reactants and/or a mixture of R²-containing reactants. Finally, the combinatorial library may be formed using a combination of these processes.

As used herein “protecting group” refers to a moiety attached to a functional group, such as an amine, to prevent an undesired reaction. Preferably the protecting group may be easily removed after protection of the functional group is no longer required. Suitable protecting groups include t-butyloxycarbonyl (Boc), 9-fluorenylmethoxycarbonyl (Fmoc), benzyloxycarbonyl, allyloxycarbonyl, and (trimethylsilyl)ethoxycarbonyl.

The present invention is directed to the synthesis of amino-substituted-TIQ-carboxylic acids, particularly amino-substituted-TIQ-carboxylic acid having the general structure:

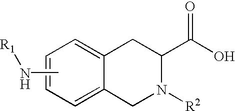

wherein R¹ and R² may be any desired moiety. Suitable R¹ include amides, sulfonamides, ureas, thioureas, alcohols, alkyls, aryls, and mixtures thereof, and suitable R² include amides, sulfonamides, ureas, thioureas, alcohols, alkyls, aryls, hydrogen and mixtures thereof

Synthetic methods in accordance with the present invention utilize a amino-substituted-tetrahydroisoquinoline-carboxylic acid attached to a solid support, preferably a resin, more preferably a polyester resin, a polyolefin resin such as polyethylene, or polyvinyl resin such as polystyrene. As used herein, the term “polyester resins” is intended to include modified polyester resins.

Methods in accordance with the invention may comprise the steps of providing a support-bound amino-substituted-tetrahydroisoquinoline-carboxylic acid, in the form of a carboxylate, wherein the ring nitrogen of the support-bound amino-substituted-tetrahydroisoquinoline has a protecting group; attaching a first moiety to the amino-substituted group of the support-bound amino-substituted-tetrahydroisoquinoline-carboxylate; de-protecting the ring nitrogen; attaching a second moiety to the ring nitrogen to form a support-bound intermediate; and cleaving the support-bound intermediate from the solid support. The steps are performed for times and at temperatures sufficient for the desired reactions to occur.

The support-bound amino-substituted-TIQ-carboxylate may be formed in any suitable manner. For example, support-bound amino-substituted-TIQ-carboxylate may be formed by providing a nitro-substituted-tetrahydroisoquinoline-carboxylic acid with a protecting group to form an orthogonally protected nitro-substituted-TIQ-carboxylic acid; attaching the orthogonally protected nitro-substituted-TIQ-carboxylic acid to the solid support; and reducing the nitro group to form the orthogonally protected amino-substituted-TIQ-carboxylate. In another embodiment, tetrahydroisoquinoline-carboxylic acid may be first bound to the support, and then may be nitrated in the 4-, 5-, 6- or 7-position followed by reduction of the nitro group to an amino group.

The TIQ-carboxylic acid may be nitrated by any suitable manner, such as treatment with sulfuric acid and potassium nitrate or with nitronium tetrafluoroborate and acetonitrile. The nitro may be reduced by any suitable manner, such as hydrogenation over a metal catalyst, preferably a palladium catalyst, SnCl₂ in dimethyl-formamide or the like. The ring nitrogen may be protected and de-protected in any suitable manner. A suitable protection method comprises reacting the TIQ-carboxylic acid with di-t-butyl bicarbonate, or with 9-fluorenylmethyl, chloroformate (Fmoc-C1), or 9-fluorenyl methoxyl carbonyl-N-hydroxy succinimide, while a suitable de-protection method comprises treatment with a strong acid such as trifluoroacetic acid or with an amine base such as piperidine. The protecting group may be selected from the group consisting of t-butyloxycarbonyl (Boc), 9-fluorenylmethoxycarbonyl (Fmoc), benzyloxycarbonyl, allyloxycarbonyl, (trimethylsilyl)ethoxycarbonyl and mixtures thereof.

Generally the TIQ-carboxylic acid is attached to the solid support by any suitable manner. In one embodiment, the TIQ-carboxylic acid is mixed with the solid support in dichloromethane in the presence of 4-dimethyl amino pyridine and 1,3-diisopropylcarbodiimide. In a preferred embodiment the TIQ-carboxylic acid is attached to the solid support through the acid moiety, more particularly through reaction of the support with the hydroxyl segment of the carboxylic acid moiety.

In one embodiment, the methods of preparing an amino-substituted-tetrahydroisoquinoline-carboxylic acid comprises the steps of: (a) providing an orthogonally protected amino-substituted-tetrahydroisoquinoline-carboxylic acid wherein the orthogonally protected amino-substituted-tetrahydroisoquinoline-carboxylic acid is attached to a solid support generally through the acid moiety and therefore is in the form of a carboxylate; (b) attaching the R¹ group to the orthogonally protected amino-substituted-tetrahydroisoquinoline-carboxylate; (c) de-protecting the orthogonally protected R¹-substituted-amino-substituted-tetrahydroisoquinoline-carboxylate to form a deprotected R¹-substituted-amino-substituted-tetrahydroisoquinoline-carboxylate; (d) attaching the R² group to the ring nitrogen of the R¹-substituted-amino-substituted-tetrahydroisoquinoline-carboxylate to form an intermediate; and (e) cleaving the intermediate from the solid support to form an R¹, R²-substituted-amino-substituted-tetrahydroisoquinoline-carboxylate. In the embodiment where R² is hydrogen, step d is optional.

The amino groups of the amino-substituted-tetrahydroisoquinoline-carboxylate (amino-substituted TIQ-carboxylate acid) provide two suitable points for attaching the R¹ and R² groups and forming disubstituted amino-substituted-TIQ carboxylate. The steps for attaching the R¹ and R² groups, respectively, occur at a temperature and for a time sufficient for the desired reactions to occur. Suitable R¹ includes amides, sulfonamides, ureas, thioureas, alcohols, alkyls, aryls, heterocyclic moieties and mixtures thereof. Suitable R² includes amides, sulfonamides, ureas, thioureas, alcohols, alkyls, aryls, heterocyclic moieties, hydrogen and mixtures thereof. Any desired moieties may be used to form the R¹ and R² groups; suitable moieties include acyl halides; carboxylic acids, including amino acids; sulfonyl chlorides; isocyanates; isothiocyanates; epoxides; halides, including alkyl halides and aryl halides; aldehydes; and mixtures thereof. As used herein “amino acids” is intended to include N-protected amino acids. The general synthetic scheme is illustrated in Reaction Sequence 1, set forth below.

Reaction Sequence 1.

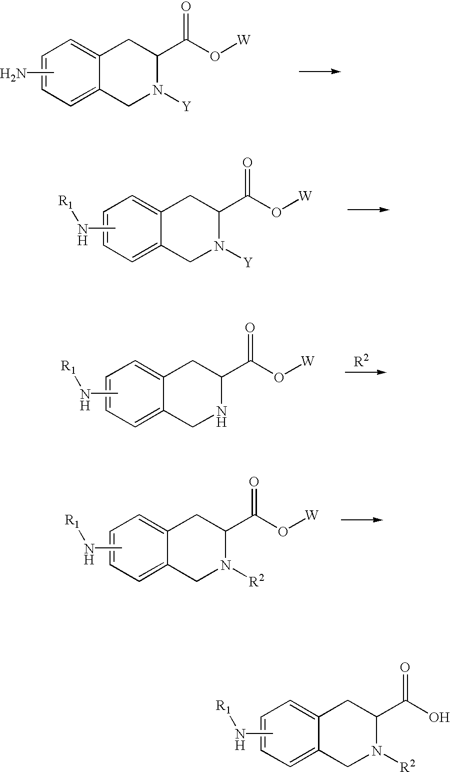

wherein W represents a solid support, Y represents a protecting group such as t-butyloxycarbonyl, and R¹ is alkyl, aryl, heterocyclic moiety, amide, sulfonamide, urea, thiourea, or alcohol; and R² is alkyl, aryl, heterocyclic moiety, amide, sulfonamide, urea, thiourea, hydrogen, or alcohol.

In one embodiment, the R¹ group and/or R² group is an amide formed by reacting the amino-substituted-TIQ-carboxylate with an acyl chloride in the presence of N,N-diisopropylethylamine (DIEPA) and dichloroethane. Preferably the reaction occurs at room temperature for a period of time of about 12 hours. In another embodiment the amide is formed by activating a carboxylic acid in solution using (benzotriazol-1-yloxy)-tris(pyrrolidino)phosphonium hexafluorophosphate (PyBOP) in dimethylformamide (DMF), and adding the solution to the amino-substituted-TIQ-carboxylate resin. Preferably the reaction occurs at room temperature for a period of time of from about 1 to about 2 hours. The carboxylic acid may be an amino acid, such as N-protected amino acid.

In another embodiment, the R¹ group and/or R² group is a sulfonamide formed by reacting the amino-substituted-TIQ-carboxylate with a sulfonyl chloride, generally in the presence of 4-(dimethylamino)pyridine (DMAP) and pyridine. Preferably the step of forming the sulfonamide comprises reacting the amino-substituted-TIQ-carboxylate with a sulfonyl chloride in the presence of about 1%, by weight, 4-dimethylaminopyridine and pyridine at room temperature for from about 6 to about 12 hours. Generally the mole ratio of amino-substituted-TIQ-carboxylate to sulfonyl chloride is from about 1:2 to about 1:8, preferably from about 1:3 to about 1:5.

In another embodiment, the R¹ group and/or R² group is a urea or thiourea formed by reacting the amino-substituted-TIQ-carboxylate with an isocyanate or isothiocyanate, respectively, generally in the presence of NaH in dimethylformamide. Preferably the amino-substituted-TIQ-carboxylate is reacted with an isocyanate or isothiocyanate in the presence of about 1%, by weight, NaH in dimethylformamide at room temperature for about 12 hours. Generally the mole ratio of amino-substituted-TIQ-carboxylate to isocyanate or isothiocyanate is from about 1:3 to about 1:5.

In another embodiment, the R¹ group and/or R² group is an alcohol formed by reacting the amino-substituted-TIQ-carboxylate with an epoxide, generally in the presence of an alcohol solvent. Preferably the reaction occurs at a temperature of about 80° C. and for a time period of about 16 hours. In one embodiment, the alcohol solvent is a mixture of ethanol and isopropanol, preferably in a volume ratio of about 1:1 ethanol:isopropanol. Generally the mole ratio of amino-substitute-TIQ-carboxylate to epoxide is from about 1:3 to about 1:5.

In another embodiment, the R¹ group and/or R² group is an alkyl formed by reacting the amino-substituted-TIQ-carboxylate with an alkyl halide, preferably an alkyl bromide. Generally the step of forming the alkyl comprises reacting the amino-substituted-TIQ-carboxylate with an alkyl halide in the presence of Bu₄NHSO₄ and Na₂CO₃. In a preferred embodiment, the step comprises reacting the amino-substituted-TIQ-carboxylate with an alkyl bromide in a solution comprising about 2%, by weight, Bu₄NHSO₄, about 5%, by weight, Na₂CO₃ and toluene at a temperature of about 70° C. for a period of time of about 8 hours. Generally the mole ratio of amino-substituted-TIQ-carboxylate to alkyl halide is from about 1:2 to about 1:4.

In one embodiment, the R¹ group and/or R² group is an alkyl formed by reductive alkylation. The amino-substituted-TIQ-carboxylate may be reacted with an aldehyde in the presence of a borane/pyridine complex. Preferably the step comprises reacting the amino-substituted-TIQ-carboxylate with an aldehyde in the presence of a borane/pyridine complex in a mixing solvent comprising ethanol and dimethyl formamide, more preferably the mixing solvent comprises ethanol and dimethyl formamide in a ethanol:dimethyl formamide weight ratio of about 3:1. Generally the mole ratio of amino-substituted-TIQ-carboxylate to aldehyde is from about 1:2 to about 1:4. The borane/pyridine complex is a commercially available reagent from Aldrich.

Preferably, the R¹ group and/or R² group may be formed by reacting the amino-substituted-TIQ-carboxylate with benzoyl chloride in the presence of N,N-diisopropylethylamine or with benzaldehyde in the presence of a borane/pyridine complex.

The support-bound intermediate may be cleaved to produce the amino-substituted-TIQ-carboxylic acid by any suitable manner. A preferred cleavage step comprises treating the support-bound intermediate with HBr. In one embodiment, the step of cleaving the intermediate from the solid support comprises treating the intermediate with a composition of from about 1% to about 2%, by weight, HBr and trifluoroacetic acid. The reaction is performed at a temperature and for a time sufficient for cleavage to occur; preferably the reaction occurs at room temperature for a period of about 24 hours. If desired, the resulting amino-substituted-TIQ-carboxylic acid may be further isolated and/or purified by any art recognized method, such as solvent extraction and recrystallization, thin layer chromatography, or high pressure liquid chromatography (HPLC).

Synthetic methods in accordance with the invention may be used to prepare combinatorial libraries of substituted amino-substituted-TIQ-carboxylic acids. In one embodiment, the invention is directed to methods of preparing a combinatorial library of amino-substituted-tetrahydroisoquinoline-carboxylic acids having the structure:

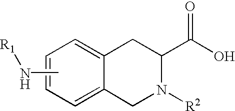

wherein R¹ is alkyl, aryl, heterocyclic moiety, amide, sulfonamide, urea, thiourea, or alcohol and R² is alkyl, aryl, heterocyclic moiety, amide, sulfonamide, urea, thiourea, hydrogen or alcohol. The methods comprise the steps of: (a) providing an orthogonally protected amino-substituted-etrahydroisoquinoline-carboxylic acid wherein the orthogonally protected amino-substituted-tetrahydroisoquinoline-carboxylic acid is attached to a solid support and therefore in the form of a carboxylate; (b) attaching a first R¹ group to a first portion of orthogonally protected amino-substituted-tetrahydroisoquinoline-carboxylate and attaching a second R¹ group to a second portion of orthogonally protected amino-substituted-tetrahydroisoquinoline-carboxylate; (c) de-protecting the first and second portions of orthogonally protected amino-substituted-tetrahydroisoquinoline-carboxylates to form first deprotected and second deprotected amino-substituted-tetrahydroisoquinoline-carboxylates; (d) attaching a first R² group to the ring nitrogen of the first deprotected amino-substituted-tetrahydroisoquinoline-carboxylate to form a first intermediate and attaching a second R² group to the ring nitrogen of the second deprotected amino-substituted-tetrahydroisoquinoline-carboxylate to form a second intermediate; and (e) cleaving the first and second intermediates from the solid support to form first and second substituted amino-substituted-tetrahydroisoquinoline-carboxylic acids.

At least one of the first R¹ group and the first R² group is different from the second R¹ group and the second R² group, respectively. The reactions for attaching the first and second R¹ groups, respectively, may be conducted separately by partitioning the orthogonally protected amino-substituted-tetrahydroisoquinoline-carboxylate into at least a first portion of orthogonally protected amino-substituted-tetrahydroisoquinoline-carboxylate and a second portion of orthogonally protected amino-substituted-tetrahydroisoquinoline-carboxylate.

Generally, R¹ is selected from the group consisting of amides, sulfonamides, ureas, thioureas, alcohols, alkyls and mixtures thereof and R² is selected from the group consisting of amides, sulfonamides, ureas, thioureas, alcohols, alkyl, hydrogen and mixtures thereof. The step of attaching the R¹ group may comprise reacting the orthogonally protected amino-substituted-tetrahydroisoquinoline-carboxylates with a reactant selected from the group consisting of acyl halides, carboxylic acids, sulfonyl chlorides, isocyanates, isothiocyanates, epoxides, alkyl halides, aldehydes, and mixtures thereof, while the step of attaching the R² group may comprise reacting the deprotected amino-substituted-tetrahydroisoquinoline-carboxylates with a reactant selected from the group consisting of acyl halides, carboxylic acids, sulfonyl chlorides, isocyanates, isothiocyanates, epoxides, alkyl halides, aldehydes, and mixtures thereof.

### EXAMPLE

A substituted 7-amino-tetrahydroisoquinoline-carboxylic acid in accordance with the invention is prepared as set forth in Reaction Sequence 2, wherein W represents a solid support, preferably a polystyrene resin, and Boc is t-butyloxycarbonyl.

Reaction Sequence 2.

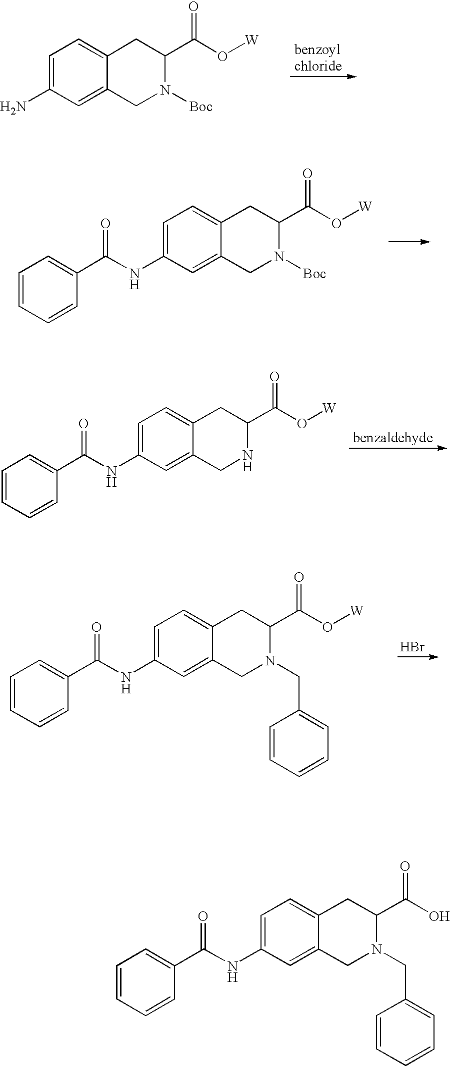

An orthogonally-protected TIQ (7-nitro-Boc-TIQ) carboxylic acid is prepared and attached to a solid support, such as a polyethylene resin. The 7-nitro group is reduced to a 7-amino group, and the support bound amino-substituted-TIQ-carboxylic acid is reacted with benzoyl chloride in the presence of N,N-diisopropylethylamine (DIEPA). After removal of the t-butyloxycarbonyl protecting group, the resulting support-bound intermediate is reacted with benzaldehyde in borane pyridine to form a di-substituted intermediate. The di-substituted intermediate is cleaved from the resin by treatment with 1% to 2%, by weight, HBr (20% by weight HBr in acetic acid solution) in trifluoroacetic acid.

Throughout the specification, all percentages and ratios are by weight unless specifically indicated otherwise. Additional embodiments and modifications within the scope of the claimed invention will be apparent to one of ordinary skill in the art. Accordingly, the scope of the present invention shall be considered in the terms of the following claims and is understood not to be limited to the details or the methods described in the specification.

What is claimed is:

1. A method of preparing an R¹, R²-substituted-amino-substituted-tetrahydroisoquinoline-carboxylic acid having the structure: 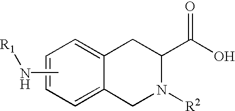
wherein R¹ is alkyl, aryl, heterocyclic moiety, amide, sulfonamide, urea, thiourea, or alcohol and R² is alkyl, aryl, heterocyclic moiety, amide, sulfonamide, urea, thiourea, alcohol, or hydrogen, the method comprising the steps of: (a) providing an orthogonally protected amino-substituted-tetrahydroisoquinoline-carboxylic acid wherein the orthogonally protected amino-substituted-tetrahydroisoquinoline-carboxylic acid is attached to a solid support and in the form of a carboxylate; (b) attaching an R¹group to the orthogonally protected amino-substituted-tetrahydroisoquinoline-carboxylate; (c) deprotecting the orthogonally protected R¹-substituted amino-substituted-tetrahydroisoquinoline-carboxylate; (d) attaching an R² group to the ring nitrogen of the deprotected R¹-substituted-amino-substituted-tetrahydroisoquinoline-carboxylate to form an intermediate attached to the solid support; and (e) cleaving the intermediate from the solid support to form an R¹, R²-substituted-amino-substituted-tetrahydroisoquinoline-carboxylic acid.

2. A method according to claim 1, wherein the step of cleaving the intermediate from the solid support comprises treating the intermediate with HBr.

3. A method according to claim 2, wherein the step of cleaving the intermediate from the solid support comprises treating the intermediate with a composition comprising from about 1% to about 2%, by weight, HBr and trifluoroacetic acid.

4. The method according to claim 1, wherein step a comprises: (i) providing a nitro-substituted-tetrahydroisoquinoline-carboxylic acid with a protecting group to form an orthogonally protected nitro-substituted-tetrahydroisoquinoline-carboxylic acid; (ii) attaching the orthogonally protected nitro-substituted-tetrahydroisoquinoline-carboxylic acid to the solid support to form the carboxylate; and (iii) reducing the nitro group to form the orthogonally protected amino-substituted-tetrahydroisoquinoline-carboxylate.

5. A method according to claim 1, wherein the protecting group is selected from the group consisting of t-butyloxycarbonyl, 9-fluorenylmethoxycarbonyl, benzyloxycarbonyl, allyloxycarbonyl, (trimethylsilyl)ethoxycarbonyl and mixtures thereof.

6. A method according to claim 1, wherein the solid support is a polystyrene resin.

7. A method according to claim 1, wherein step b comprises reacting the orthogonally protected amino-substituted-tetrahydroisoquinoline-carboxylate with benzoyl chloride in the presence of N,N-diisopropylethylamine.

8. A method according to claim 1, wherein step d comprises reacting the R¹-substituted-amino-substituted-tetrahydroisoquinoline-carboxylate with benzaldehyde in a borane/pyridine complex.

9. A method according to claim 1, wherein the step of attaching the R¹ group comprises a step selected from the group consisting of: (i) reacting the orthogonally protected amino-substituted-tetrahydroisoquinoline-carboxylate with an acyl chloride in the presence of N,N-diisopropylethylamine and dichloroethane; (ii) activating a carboxylic acid in solution using (benzotriazol-1-yloxy)-tris(pyrrolidino)phosphonium hexafluorophosphate in dimethylformamide, and adding the solution to the orthogonally protected amino-substituted-tetrahydroisoquinoline-carboxylate; (iii) activating a N-protected amino acid in solution using (benzotriazol-1-yloxy)-tris(pyrrolidino)phosphonium hexafluorophosphate in dimethylformamide, and adding the solution to the orthogonally protected amino-substituted-tetrahydroisoquinoline-carboxylate; (iv) reacting the orthogonally protected amino-substituted-tetrahydroisoquinoline-carboxylate with a sulfonyl chloride in the presence of 4-dimethylaminopyridine and pyridine; (v) reacting the orthogonally protected amino-substituted-tetrahydroisoquinoline-carboxylate with an isocyanate in the presence of NaH in dimethylformamide; (vi) reacting the orthogonally protected amino-substituted-tetrahydroisoquinoline-carboxylate with an isothiocyanate in the presence of NaH in dimethylformamide; (vii) reacting the orthogonally protected amino-substituted-tetrahydroisoquinoline-carboxylate with an epoxide in the presence of an alcohol solvent; (viii) reacting the orthogonally protected amino-substituted-tetrahydroisoquinoline-carboxylate with an alkyl halide in the presence of Bu₄NHSO₄ and Na₂CO₃; and (ix) reacting the orthogonally protected amino-substituted-tetrahydroisoquinoline-carboxylate with an aldehyde in the presence of a borane/pyridine complex.

10. A method according to claim 1, wherein the step of attaching the R² group comprises a step selected from the group consisting of: (i) reacting the R¹-substituted-amino-substituted-tetrahydroisoquinoline-carboxylate with an acyl chloride in the presence of N,N-diisopropylethylamine and dichloroethane; (ii) activating a carboxylic acid in solution using (benzotriazol-1-yloxy)-tris(pyrrolidino)phosphonium hexafluorophosphate in dimethylformamide, and adding the solution to the R¹-substituted-amino-substituted-tetrahydroisoquinoline-carboxylate; (iii) activating a N-protected amino acid in solution using (benzotriazol-1-yloxy)-tris(pyrrolidino)phosphonium hexafluorophosphate in dimethylformamide, and adding the solution to the R¹-substituted-amino-substituted-tetrahydroisoquinoline-carboxylate; (iv) reacting the R¹-substituted-amino-substituted-tetrahydroisoquinoline-carboxylate with a sulfonyl chloride in the presence of 4-dimethylaminopyridine and pyridine; (v) reacting the R¹-substituted-amino-substituted-tetrahydroisoquinoline-carboxylate with an isocyanate in the presence of NaH in dimethylformamide; (vi) reacting the R¹-substituted-amino-substituted-tetrahydroisoquinolin-carboxylate with an isothiocyanate in the presence of NaH in dimethylformamide; (vii) reacting the R¹-substituted-amino-substituted-tetrahydroisoquinoline-carboxylate with an epoxide in the presence of an alcohol solvent; (viii) reacting the R¹-substituted-amino-substituted-tetrahydroisoquinoline-carboxylate with an alkyl halide in the presence of Bu₄NHSO₄ and Na₂CO₃; and (ix) reacting the R¹-substituted-amino-substituted-tetrahydroisoquinoline-carboxylate with an aldehyde in the presence of a borane/pyridine complex.

11. A method according to claim 1, wherein the amino-substituted-tetrahydroisoquinoline-carboxylic acid is 7-amino-tetrahydroisoquinoline-carboxylic acid.

12. A method of preparing a combinatorial library of amino-substituted-tetrahydroisoquinoline-carboxylic acids having the structure: 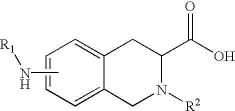
wherein R¹ is alkyl, aryl, heterocyclic moiety, amide, sulfonamide, urea, thiourea, or alcohol, and R² is alkyl, aryl, heterocyclic moiety, amide, sulfonamide, urea, thiourea, alcohol or hydrogen, the method comprising the steps of: (a) providing an orthogonally protected amino-substituted-tetrahydroisoquinoline-carboxylic acid wherein the orthogonally protected amino-substituted-tetrahydroisoquinoline-carboxylic acid is attached to a solid support and in the form of a carboxylate; (b) attaching a first R¹ group to a first portion of the orthogonally protected amino-substituted-tetrahydroisoquinoline-carboxylate and attaching a second R¹ group to a second portion of the orthogonally protected amino-substituted-tetrahydroisoquinoline-carboxylate; (c) deprotecting the first and second portions of the orthogonally protected amino-substituted-tetrahydroisoquinoline-carboxylates to form first and second deprotected amino-substituted-tetrahydroisoquinoline-carboxylates; (d) attaching a first R² group to the ring nitrogen of the first deprotected amino-substituted-tetrahydroisoquinoline-carboxylate to form a first intermediate and attaching a second R² group to the ring nitrogen of the second deprotected amino-substituted-tetrahydroisoquinoline-carboxylate to form a second intermediate; and (e) cleaving the first and second intermediates from the solid support to form a first and second R¹, R²-substituted-amino-substituted-tetrahydroisoquinoline-carboxylic acids; wherein at least one of the first R¹ group and the first R² group is different from the second R¹ group and the second R² group, respectively.

13. A method according to claim 12, wherein the step of cleaving the first and second intermediates from the solid support comprises treating the intermediates with HBr.

14. A method according to claim 12, wherein the amino-substituted-tetrahydroisoquinoline-carboxylic acid is 7-amino-tetrahydroisoquinoline carboxylic acid.

15. A method of preparing an amino-substituted-tetrahydroisoquinoline-carboxylic acid comprising the steps of: (a) providing a support-bound amino-substituted-tetrahydroisoquinoline compound having the structure: 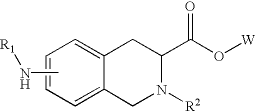
wherein R¹ is alkyl, aryl, heterocyclic moiety, amide, sulfonamide, urea, thiourea, or alcohol, and R² is alkyl, aryl, heterocyclic moiety, amide, sulfonamide, urea, thiourea, hydrogen, or alcohol, and W represents a support, and (b) cleaving the support-bound amino-substituted-tetrahydroisoquinoline compound to form an amino-substituted-tetrahydroisoquinoline-carboxylic acid.

16. A method according to claim 15, wherein the step of cleaving the intermediate from the solid support comprises treating the intermediate with HBr.

17. A method according to claim 15, wherein the solid support is a polystyrene resin.

18. A method according to claim 15, wherein the amino-substituted-tetrahydroisoquine-carboxylic acid is 7-amino-tetrahydroisoquinoline carboxylic acid.

19. A method of preparing an amino-substituted-tetrahydroisoquinoline-carboxylic acid comprising the steps of: (a) providing a support-bound amino-substituted-tetrahydroisoquinoline-carboxylic acid in the form of a carboxylate, wherein the ring nitrogen of the support-bound amino-substituted-tetrahydroisoquinoline has a protecting group; (b) attaching a first moiety to the 7-amino group of the support-bound amino-substituted-tetrahydroisoquinoline-carboxylate having a protecting group; (c) removing the protecting group from the ring nitrogen of the support-bound amino-substituted-tetrahydroisoquinoline-carboxylate; (d) attaching a second moiety to the ring nitrogen of the support-bound amino-substituted-tetrahydroisoquinoline-carboxylate to form a support-bound intermediate; and (e) cleaving the support-bound intermediate from the solid support to form an amino-substituted-tetrahydroisoquinoline-carboxylic acid.

20. A method according to claim 19, wherein the step of cleaving the support-bound intermediate from the solid support comprises treating the intermediate with a composition comprising HBr and trifluoroacetic acid.

21. A method according to claim 19, wherein the solid support is a resin.

22. A method according to claim 19, wherein the amino-substituted-tetrahydroisoquinoline-carboxylic acid is 7-amino-tetrahydroisoquinoline-carboxylic acid.
